(19) United States
(12) Patent Application Publication
(10) Pub. No.: US 2011/0319795 A1
(43) Pub. Date: Dec. 29, 2011

(54) METHOD AND SYSTEM FOR GENERATING LOCAL HEAT

(75) Inventors: Yuri Lechtzier, Eiazar (IL); Michael Kardosh, Kiryat-Ono (IL)

(73) Assignee: Applisonix Ltd., Rechovot (IL)

(21) Appl. No.: 13/255,243

(22) PCT Filed: Mar. 3, 2010
(86) PCT No.: PCT/IL10/00176

§ 371 Date: Sep. 8, 2011

Related U.S. Application Data
(60) Provisional application No. 61/158,433, filed on Mar. 9, 2009.
(60) Provisional application No. 61/272,179, filed on Aug. 27, 2009.

(51) Int. Cl. A61N 7/02

(52) U.S. Cl. 601/3

(57) ABSTRACT

A system for generating heat at a target location is disclosed. The system comprises a fiber device having a distal end and a proximal end, an acoustic wave generator for generating acoustic waves, and an acoustic coupler for coupling acoustic energy carried by the acoustic waves into the distal end such that heat is generated at the distal end.

### RELATED APPLICATION/S

This application claims the benefit of priority from U.S. Patent Application No. 61/158,433 filed Mar. 9, 2009 and U.S. Patent Application No. 61/272,179 filed Aug. 27, 2009. The contents of all of the above documents are incorporated by reference as if fully set forth herein.

### FIELD AND BACKGROUND OF THE INVENTION

The present invention, in some embodiments thereof, relates to medicine and, more particularly, but not exclusively, to a method and system for generating local heat.

Generation of heat for treating a biological tissue or lymph node, e.g., to stop hemorrhage, is known. Various devices may be used for the purpose of producing heat. Laser devices, microwave and radiofrequency (RF) antennas and thermal fluids are commonly used.

For example, European patent No. EP 0 370 890 discloses a device which includes a microwave antenna enclosed in a catheter. The antenna is designed to emit electromagnetic energy to the tissue surrounding the antenna. The catheter is also equipped with cooling channels for cooling of the tissue closest to the catheter.

U.S. Pat. No. 5,366,490 discloses means of treatment in which the needle is advanceable so as to exit the catheter. The catheter and the needle are controlled in place with the aid of an ultrasound imaging device, which during the entire treatment continuously monitors the area of treatment.

U.S. Pat. No. 5,257,977 discloses a catheter is provided with a reservoir for fluid. The reservoir is flexible and is connected via channels through the catheter with a heating device located outside the body. The fluid is heated in the heating device and circulated through the channels and the reservoir that to some degree expands for better contact with the tissue. The rise of temperature in the reservoir also brings about heating of the surrounding tissue. Treatment is affected by controlling the temperature of the circulating fluid.

International Patent Publication No. 97/02794 discloses a heating device contained inside an expandable reservoir. The heating device is provided with energy from an assembly outside of the body for heating of fluid inside the reservoir. The heating device is designed as a resistance wire or similar and heats the fluid through convection.

U.S. Pat. No. 4,646,756 discloses an ultrasound hyperthermia unit which includes an ultrasound transducer angled to direct ultrasound energy towards an acoustic focus and a temperature sensor which provide an output signal indicative of temperature values at the focus. The output signal controls the power output and the position of the acoustic focus to achieve localized heating of tumor tissues above viability. Both the shape and power of the acoustic focus is adjusted to take account of the density and shape of neoplastic tissues.

### SUMMARY OF THE INVENTION

According to an aspect of some embodiments of the present invention there is provided a system for generating heat at a target location. The system comprises a fiber device having a distal end and a proximal end, an acoustic wave generator for generating acoustic waves, and an acoustic coupler for coupling acoustic energy carried by the acoustic waves into the distal end such that heat is generated at the distal end.

According to some embodiments of the invention the invention the system comprises an acoustic amplifier configured for amplifying amplitude of the acoustic waves prior to the coupling of the acoustic energy into the fiber device.

According to some embodiments of the invention the acoustic amplifier comprises an acoustic horn.

According to some embodiments of the invention the acoustic coupler comprises a container filled with an impedance matching medium.

According to some embodiments of the invention the container comprises a first end and a second end, wherein the acoustic wave generator is coupled to the container at the first end, and wherein the second end comprises an opening which receives the proximal end of the fiber device.

According to some embodiments of the invention a height of the at least one opening is approximately an integer multiplication of half a wavelength of the ultrasound waves.

According to some embodiments of the invention the acoustic coupler comprises a mechanical gripper having gripping ends biasable towards each other, wherein the acoustic wave generator induces vibrations at least at one of the gripping ends, and wherein the fiber device is positioned between the gripping ends to receive the vibrations.

According to some embodiments of the invention the at least one gripping end is sufficiently flexible and sufficiently elastic such that amplitude of the vibrations is larger at the at least one gripping end than at the acoustic wave generator.

According to some embodiments of the invention the acoustic coupler comprises to an acoustic horn and wherein the at least one gripping end and the acoustic horn are made of the same material.

According to some embodiments of the invention the at least one gripping end is an integral extension of the acoustic horn.

According to an aspect of some embodiments of the present invention there is provided a method of generating heat at a target location. The method comprises: guiding a distal end of a fiber device to the target location and coupling acoustic energy into the fiber device at a proximal end of the fiber device such as to generate heat at the distal end, thereby generating heat at the target location.

According to some embodiments of the invention the heat is generated at an amount being sufficient to treat a target tissue at the target location.

According to some embodiments of the invention the target tissue is a tumor. According to some embodiments of the invention the target tissue is a lesion. According to some embodiments of the invention the target tissue is an inflammatory tissue. According to some embodiments of the invention the target tissue is a skin tissue. According to some embodiments of the invention the target tissue is part of an internal organ. According to some embodiments of the invention the target tissue is a blood vessel clot.

According to some embodiments of the invention the guiding is via a minimally invasive procedure.

According to some embodiments of the invention the guiding is during an invasive procedure.

According to some embodiments of the invention the method comprises amplifying the amplitude of acoustic waves carrying the acoustic energy prior to the coupling of the acoustic energy into the fiber device.

According to some embodiments of the invention the fiber device comprises a bulb at its distal end.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram describing a method suitable for generating heat at a target location, in various exemplary embodiments of the invention;

FIG. 2A is a schematic illustration showing exploded view of a system for generating heat at a target location, according to various exemplary embodiments of the present invention;

FIG. 2B is a schematic illustration of a portion of a fiber device, according to various exemplary embodiments of the present invention;

FIG. 3 is a schematic illustration of a system for generating heat at a target location in embodiments of the invention in which the system comprises a container filled with an impedance matching medium;

FIGS. 4A-B are schematics illustration of a system for generating heat at a target location in embodiments in which the acoustic energy is delivered by direct vibration;

FIGS. 5A-B show an thermal image captured by an infrared camera (FIG. 5A), and a temperature graph (FIG. 5B) describing a fiber shaft without a bulb that was excited by acoustic waves, as obtained in experiments performed according to some embodiments of the present invention;

FIG. 6 shows an thermal image captured by an infrared camera describing a fiber shaft which comprises a bulb excited by acoustic waves, as obtained in experiments performed according to some embodiments of the present invention; and

FIG. 7 shows an thermal image captured by an infrared camera describing a fiber shaft which comprises a bulb excited by acoustic waves, as obtained in experiments performed according to some embodiments of the present invention for a case in which the bulb is made of a material with higher mechanical energy absorption and lower thermal conductivity compared to the material of the shaft.

### DESCRIPTION OF SPECIFIC EMBODIMENTS OF THE INVENTION

The present invention, in some embodiments thereof, relates to medicine and, more particularly, but not exclusively, to a method and system for generating local heat.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present inventors found that when acoustic energy, preferably ultrasound energy, is coupled into a fiber, the end of the fiber is heated. The present inventors also found that due the changes in the physical property of the fiber during heating, the amount of heat that is generated at the end of the fiber can be significantly higher than the amount of heat that is generated along the fiber. By analyzing the equation governing the dynamic of acoustic waves in a fiber, the present inventors uncovered that once the temperature of the fiber begins to rise, the locations of hot spots along the fiber are shifted toward the end of the fiber. The present inventors discovered that by judicious selection of the shape and properties of the fiber's end the temperature of hot spots along the fiber can be significantly reduced, and in some embodiments becomes close or equal to environmental temperature, while generating a significant amount of heat at the end of fiber.

The present embodiments utilize a fiber as a medium for carrying acoustic energy for the purpose of heating a target location. In various exemplary embodiments of the invention the target location is in or on a living and the acoustic energy heats the tissue at the target location. Thus the present embodiments can be utilized for treating a tissue, typically a diseased or abnormal tissue, by inducing hyperthermia to the tissue.

The present inventors found that hyperthermia is effective in treating diseases like cancerous growths. The therapeutic benefit of hyperthermia therapy is mediated through the principal that a directly tumouricidal effect on tissue by raising temperatures to above than 42° C. results in irreversible damage to the cells. The lack of any cumulative toxicity associated with hyperthermia therapy, in contrast to other types of therapy, such as radio- and chemotherapy, is further justification for seeking to develop improved systems for hyperthermia therapy.

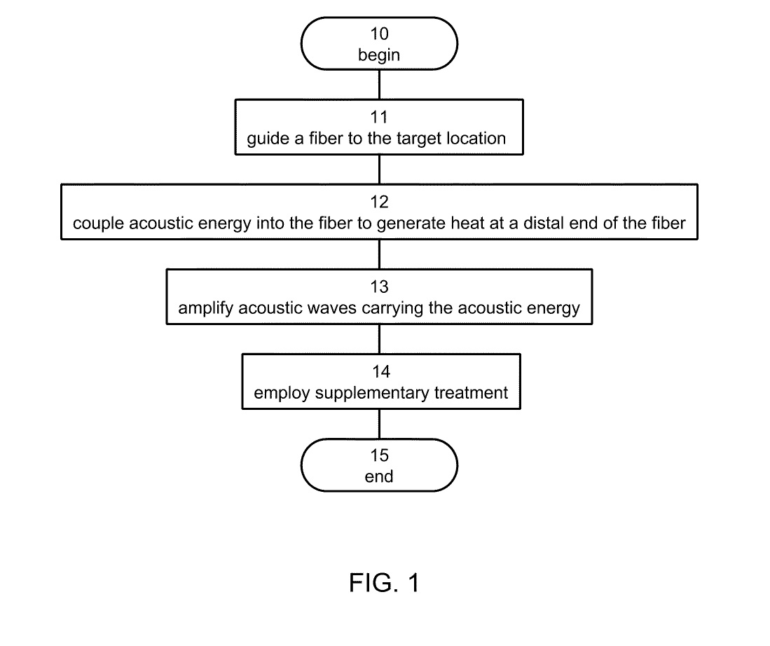

Referring now to the drawings, FIG. 1 is a flowchart diagram describing a method suitable for generating heat at a target location, in various exemplary embodiments of the invention.

It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The method begins at 10 and continues to 11 at which a distal end of a fiber device is guided to a target location, typically until a contact is established between the distal end and an object located at the target location. In some embodiments, the target location is in or on a living body, such as a mammal (e.g., a human), in which case the object at the target location is a target tissue. The target tissue can be, for example, a tumor, a lesion, an inflammatory tissue or the like, and it can be located on the skin, beneath the skin, in a body-fluid vessel (e.g., blood vessel, urine vessel), or in another internal organ.

The fiber device can be guided to the target location in any way known in the art. For example, when the target location is in a living body the fiber device can be guided during a minimally invasive procedure, such as a laparoscopic procedure or an endoscopic procedure. The guiding can be facilitated by a catheter as known in the art. In some embodiments, the fiber device is monitored during guidance, for example, using an ultrasound imaging system, magnetic resonance imaging system, X ray imaging system or the like.

The fiber device can be guided to the target location during a fully invasive procedure, i.e., open surgery. When the target location is on the skin of the living body, the fiber device can be placed directly on the target location.

The method continues to 12 at which acoustic energy is coupled into the fiber device at a proximal end thereof such as to generate heat at the distal end of the fiber device. In various exemplary embodiments of the invention the heat is generated at an amount which sufficient to treat the target tissue, e.g., by inducing hyperthermia.

In some embodiments of the present invention the method continues to 13 in which an amplitude of acoustic waves carrying the acoustic energy is amplified prior to the coupling of acoustic energy into the fiber device. Techniques suitable for such amplifications are provided hereinunder.

The method can optionally and preferably continue to 14 at which a supplementary treatment is employed to the target tissue. Such supplementary treatment can include radiotherapy, brachytherapy, chemotherapy or any combination thereof.

It is expected that during the life of a patent maturing from this application many relevant treatments will be developed and the scope of the term supplementary treatment is intended to include all such new technologies a priori.

It is envisioned that hyperthermia and the supplementary treatment will be synergistic. For example, it is recognized that even small fractions of a degree of temperature variation can significantly alter the prospects of cells surviving a radiation insult. Factors affecting the synergistic action of hyperthermia and the supplementary treatment include, but are not limited to, the degree of duration of hyperthermia, the sequence of hyperthermia and the supplementary treatment, the fractionated and total dose of the supplementary treatment, the pH of the extra-cellular milieu, the nutrient status of cells and the histological type and malignant status of the cells.

The method ends at 15.

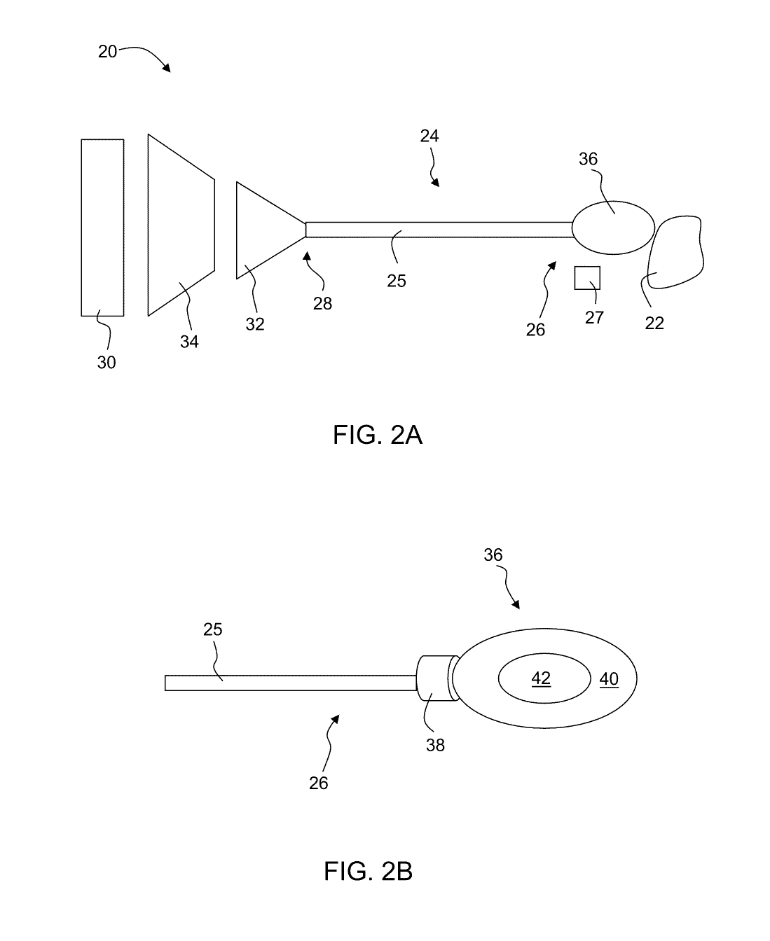

Reference is now made to FIG. 2A which is a schematic illustration showing exploded view of a system 20 for generating heat at a target location 22, according to various exemplary embodiments of the present invention. System 20 comprises showing a fiber device 24 which comprises a fiber shaft 25 having a distal end 26 and a proximal end 28. Fiber device 24 can be made of any solid material suitable for conveying acoustic energy. Preferably, fiber device 24 is made of a biocompatible material, including, without limitation, a metal and a polymer. Nonmetallic materials, such as silicon are also contemplated. The fiber device can be of any length from a few millimeters to a few meters. The diameter of the can fiber device be from about 50 μm to about 2 mm. In various exemplary embodiments of the invention the diameter of the fiber is significantly smaller (e.g., at least 10 times or at least 100 times smaller) than its length.

System 20 further comprises an acoustic wave generator 30 which generating acoustic waves. Acoustic wave generator 30 can be embodied in the form as electromechanical transducer which typically comprises a piezoelectric element. In various exemplary embodiments of the invention acoustic wave generator 30 generates vibration at an ultrasound frequency. In various exemplary embodiments of the invention the characteristic wavelength of the acoustic wave within the fiber is significantly larger (e.g., at least 10 times larger) than the diameter of the fiber.

System 20 further comprising an acoustic coupler 32 which couples acoustic energy carried by the acoustic waves into distal end 28 of fiber device 24 such that heat is generated at distal end 26. In some embodiments of the present invention system 20 comprises an acoustic amplifier 34 which is configured for amplifying the amplitude of acoustic waves prior to the coupling of acoustic energy into the fiber device. The acoustic amplifier can comprise an acoustic horn, which is constructed to receive vibrations from generator 30 and transmit the vibrations to acoustic coupler 32. Preferably, the horn is tapered toward acoustic coupler 32. In some embodiments, the horn is a stepped horn as known in the art. Additional types of acoustic amplifiers suitable for the present invention are provided hereinunder.

FIG. 2B is a schematic illustration of the distal end of fiber device 24, according to various exemplary embodiments of the present invention. Distal end 26 comprises a tip 36 which in some embodiments has a shape of a bulb. In some embodiments of the present invention the length of the bulb along the longitudinal direction of the fiber is shorter than the wavelength of the acoustic wave. Denoting wavelength by λ, the length of the bulb along the longitudinal direction of the fiber is preferably less than 0.5λ, or less than 0.1λ.

Optionally and preferably the bulb is made of a material with higher mechanical energy absorption coefficient and/or lower thermal conductivity compared to the material of the shaft. It was found by the present inventor that with such configuration the temperature of the fiber is significantly lower than the temperature of the bulb. In various exemplary embodiments of the invention the temperature of the bulb is at least 10° C. or at least 20° C. higher than the highest temperature along the shaft.

Tip 36 may be embodied as an acoustic resonator. For example, in some embodiments of the present invention tip 36 has a core-shell structure in which the core 42 is made of a different material than the shell 40. Preferably, the density of the material is lower in the core than in the shall. Also contemplated, are embodiments in which the core is gaseous (for example, an inert gas). A representative example is a core which is made of an inert gas and a shall which is made of an silicon rubber.

Optionally, fiber device 24 also comprises a matching element 38 between shaft 25 and tip 36. Matching element 38 serves for enhancing impedance coupling between shaft 25 and tip 36. For example, matching element 38 can be made of a material whose acoustic impedance is between the acoustic impedance of the shaft and the acoustic impedance of the tip.

In use, wave generator 30 vibrates, preferably at ultrasound frequency, and generates the acoustic waves in the medium adjacent thereto. In embodiments in which acoustic amplifier 34 is employed, the acoustic waves are received amplifier 34 which amplifies the amplitude of the vibrations. Coupler 32 receives the acoustic waves and couples the acoustic energy carried thereby into distal end 28 of fiber device 24. Fiber shaft 25 begins to vibrate at the vibration frequency, and, similarly to a solid rod, a longitudinal standing wave is generated in shaft 25. The standing wave results in efficient propagation of acoustic energy through shaft 25 and into tip 36 resulting in heating of the tip. The phenomena associated with acoustic wave in a fiber shaft are explained in more details in the Examples section that follows. In various exemplary embodiments of the invention the tip is heated to a temperature which is above 42° C. or above 44° C. or above 46° C. or above 48° C. or above 50° C.

System 20 can be a standalone system or it can be embodied for specific application. For example, in some embodiments, system 20 is manufactured as gastroscope, in some embodiments, system 20 is manufactured as an a endoscope, and in some embodiments system 20 is manufactured as an a laproscope.

In some embodiments of the present invention system 20 comprises a temperature sensor 27 positioned near the distal end 26. Sensor 27 senses the temperature at target location 22 and transmits it to a monitoring system (not shown) for monitoring the treatment. Temperature sensor 27 can be mounted on fiber shaft 25 or on a catheter (not shown) through which shaft 25 is guided to target location 22.

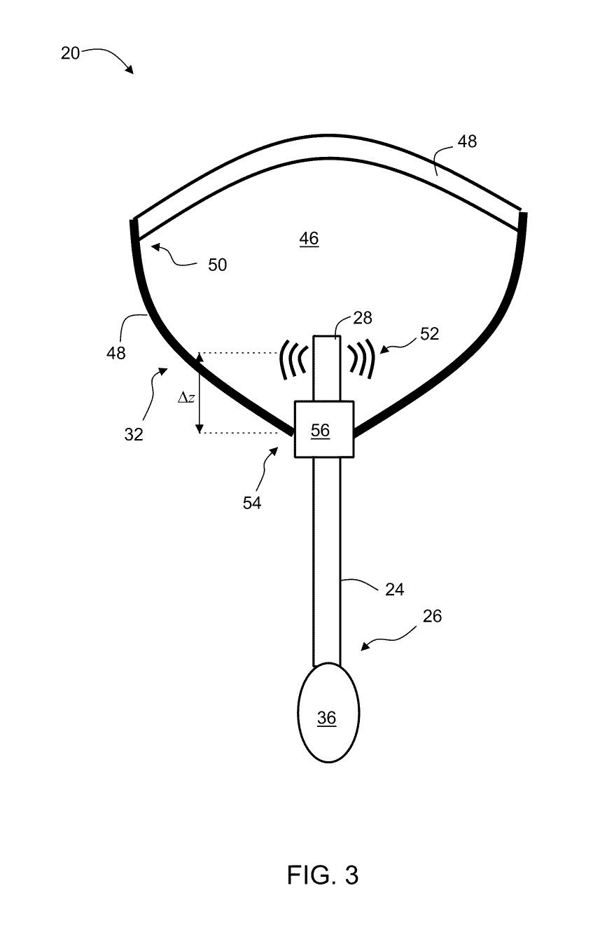

FIG. 3 is a schematic illustration of system 20 in embodiments in which acoustic coupler 32 comprises a container 44 filled with an impedance matching medium 46, such as an ultrasonic fluid or ultrasonic gel or the like. An electromechanical transducer element 48 is coupled to a container 44 at one of its ends, referred to as the proximal end 50, such that ultrasound waves are generated by element 48 in medium 46. Transducer element 48 can be planar or it can have a curvature, as desired. Container 44 preferably has a curved shape selected to focus the ultrasound waves to a focus region 52 of high acoustic pressure, at a distance Δz from an end 54 of container 44 which is distal with respect to element 48.

For example, container can have a shape of a tapered frustum (e.g., conical frustum, pyramidal frustum) having a large-area base and a small-area base, wherein transducer element 48 is coupled to the large-area base of the frustum. Focus region 52 may be a focal spot but, more preferably, focus region 52 is a locus of focal spots of ultrasound energy. For example, focus region 52 may be a substantially planar locus at some distance from transducer element 48.

In various exemplary embodiments of the invention acoustic coupler 32 comprises shaft receiving element 56 covering container 48 at end 54. When container 48 is shaped as a tapered frustum, element 56 preferably covers the small-area base of the frustum. Element 56 is preferably formed as a neck (e.g., a hollow cylinder) and constituted to receive shaft 25 such that distal end 28 enters the volume encapsulated by container 44 and contacts medium 46.

It was found by the present Inventors that focus region 52 can be formed in close proximity to element 56 when the height of element 56 is approximately an integer multiplication of half the wavelength of the ultrasound wave generated by element 48. Such neck is referred to herein as “resonant neck.” In various exemplary embodiments of the invention the height of the neck is approximately λ/2, where λ is the wavelength of the ultrasound wave. In some embodiments of the present invention the height of the neck is approximately λ, and in some embodiments the height of the neck is approximately 1.5λ. In such construction, a standing wave is formed within the neck and the focus region is formed near the neck. The shaft pass through focus 52 region and the high pressure at region 52 induces in the shaft longitudinal ultrasound waves which generate heat at distal end 26 as further detailed hereinabove. In various exemplary embodiments of the invention the diameter of the neck is substantially smaller than the wavelength of the ultrasound wave.

In embodiments in which the acoustic coupler comprises a container filled with an impedance matching medium, generator 30 preferably vibrates at an ultrasound frequency of from about 250 kHz to about 2.5 MHz. In any of these embodiments, the focused region of high acoustic pressure is formed at a distance Δz of less than 2 mm, more preferably less than 1.5 mm, more preferably less than 1 mm, more preferably less than 0.5 mm, from the inwardly facing end of the opening which receives the shaft of the fiber device.

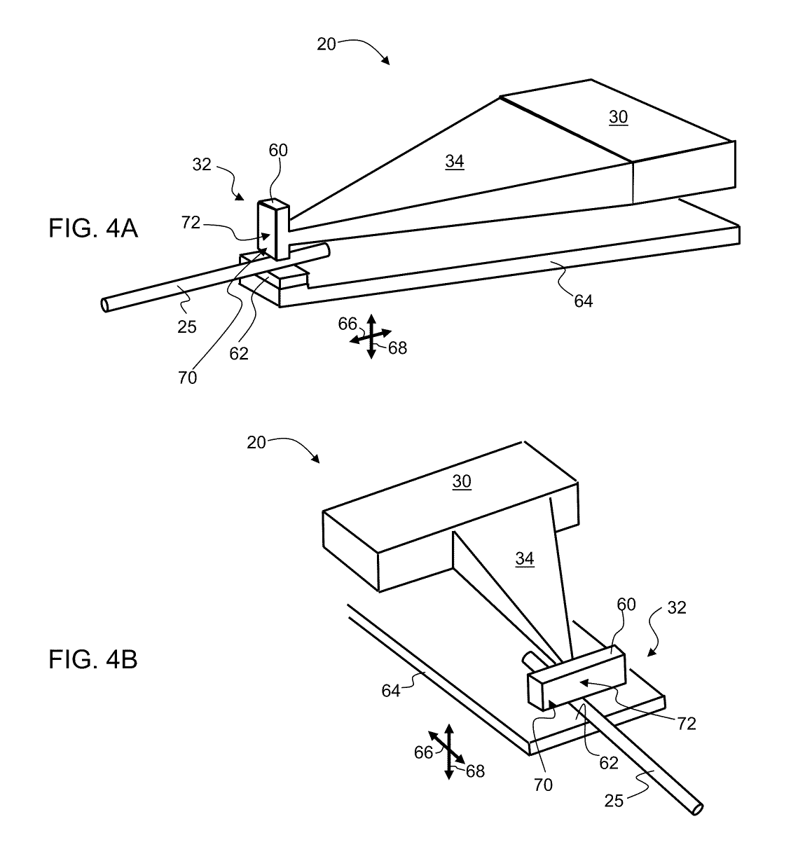

FIGS. 4A-B are schematics illustration of system 20 in embodiments in which the acoustic energy is coupled into shaft 25 by direct vibration of the shaft. In these embodiments, acoustic coupler comprises a mechanical gripper having gripping ends 60, 62 biasable towards each other. Shaft 25 of fiber device 24 is positioned between gripping ends 60, 62. In these embodiments, acoustic wave generator 30 induces vibrations at least at one of the gripping ends and shaft 25 receives the vibrations. In the representative illustrations of FIGS. 4A-B, one of the gripping ends (gripping end 60 in the present example) receives the vibrations via acoustic amplifier 34 which is embodied as a horn in the present example. The horn is preferably tapered toward gripping end 60. In some embodiments of the present invention acoustic amplifier 34 and gripping end 60 are made of the same material, and in some embodiments gripping end 60 is an integral extension of acoustic amplifier 34.

Gripping end 62 is connected to an elongated member 64 which is static in the present example. However, this need not necessarily be the case, since, for some applications, it may not be necessary for one of the gripping ends to be static. In some embodiments, both griping ends vibrate.

Generator 30, amplifier 34 and gripping end 60 can be viewed collectively as a vibratory unit or vibratory tong, and gripping end 62 and elongated member 64 can be viewed collectively as a biasing unit or biasing tong. The vibrating tong serves as a “hammer” and is preferably mounted in a cantilever fashion. The biasing tong serves as an “anvil” and can have the shape of a planar surface, which may be slanted or parallel to the vibrating tong.

In operation, the two tongs are pressed against each other and shaft 25 is gripped between ends 60 and 62. The two tongs can be pre-stressed loaded by a force that biases the two inturned surfaces of ends 60 and 62 towards each other. When generator 30 is activated, vibrations are transmitted, optionally through amplifier 34 to gripping end 60 which in turn transmits the vibrations to shaft 25. These vibrations generate a longitudinal ultrasound wave in shaft 25, which ultrasound wave generates heat at the target location (not shown in FIGS. 4A-B).

Generator 30 may be constructed so as to induce vibratory displacements of gripping end 60 along the longitudinal axis of shaft 25, as shown by arrow 66, or perpendicularly to shaft 25, as shown by arrow 68. Also contemplated, are vibratory motions which are a combination of motion along the longitudinal axis of the shaft and motion perpendicularly to the shaft.

Optionally and preferably, the amplitude of the vibrations are further amplified by the vibrating gripping end 60. This can be done, for example, by providing gripping end 60 with sufficient flexibility and elasticity such that the amplitude of the vibrations is enhanced by an elastic resonance effect. In some embodiments, gripping end 60 has an elongated shape, and amplifier 34 is coupled to the center 72 or near the center of gripping end 60. The elongation of gripping end 60 is typically perpendicular to shaft 25, either in parallel (FIG. 4B) or perpendicularly (FIG. 4A) to member 64. The amplitude of the vibrations is thus enhanced at the off-center parts 70 of gripping end 60. Optionally and preferably the amplitude of the vibrations of gripping end 60 (particularly at its off-center parts) is larger than the amplitude of the vibrations of generator 30 and amplifier 34. In various exemplary embodiments of the invention the amplitude of the vibratory displacement at the off-center parts of gripping end 60 are X times larger than the amplitude of the vibratory displacement at the contact between amplifier 34 and gripping end 60, where X is can be any positive number larger than 1, e.g., at least 1.5, more preferably at least 2, more preferably at least 4, more preferably at least 6, more preferably at least 8, more preferably at least 10.

In embodiments in which a longitudinal ultrasound wave is generated in the hair shaft by direct vibration, the acoustic wave generator preferably vibrates at an ultrasound frequency of from about 100 kHz to about 250 kHz. The mechanical vibration at the contact between the shaft of the fiber device and the gripping element are preferably at amplitude of from about 5 μm to about 50 μm.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Theoretical Considerations

Some embodiments of the present invention are based on physical phenomena that occur in a fiber with a bulb when exposed to an ultrasonic wave. In the following computation model, the fiber shaft is represented as a solid rod and the bulb is represented as a knob at the bottom of the rod. The source of vibration is located close to the top of the rod.

Excitation of the fiber by mechanical vibrations causes a quasi-standing longitudinal wave in the fiber. Acoustic energy that is absorbed by the fiber results in heating of the fiber. The absorption rate at the ultrasound frequency range, particularly from about 100 kHz to about 2.5 MHz, is typically such that the heating of the fiber shaft is available only close to points of strain energy maximum amplitude (also known as the power peaks of the standing wave).

In an ideal model of a standing wave without energy losses, there is no energy flux along the rod. In the present embodiments, however, some acoustic energy is transferred to heat, particularly at the power peaks, and there is an energy flux along the rod. In other words, there are hot spots along the rod at the location of the power peaks. The stress-strain phase angle in the nodes does not equal zero.

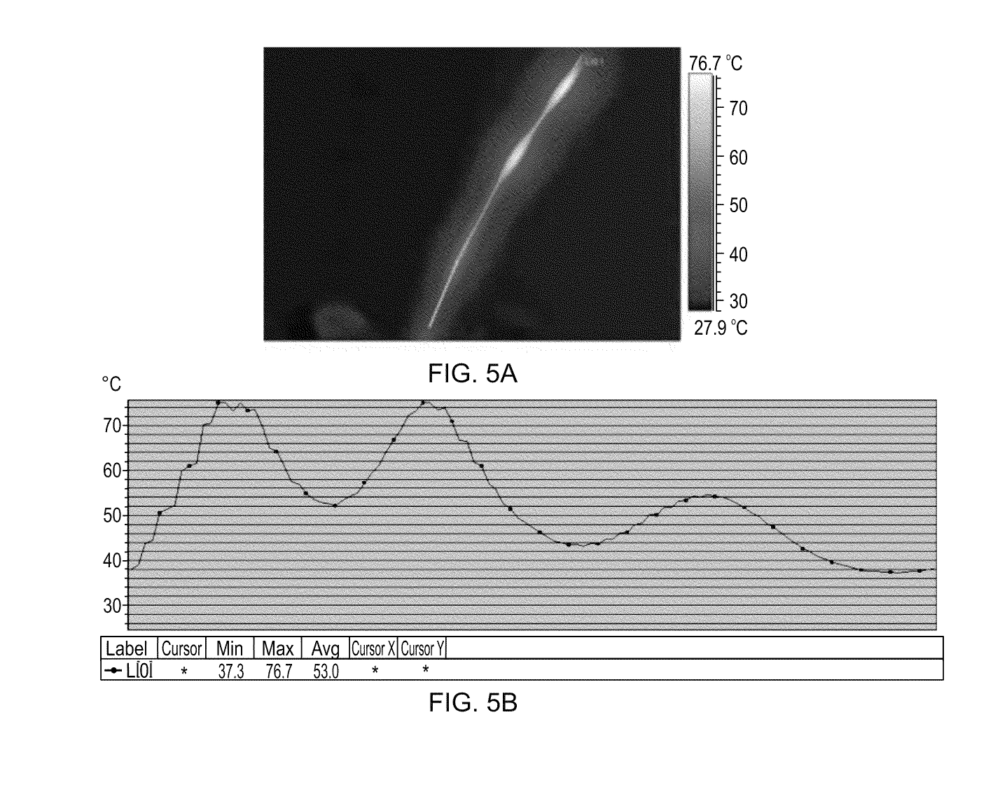

A typical thermal image of quasi-standing waves in nonmetallic fiber shaft at the aforementioned frequency is shown in FIG. 5A. The corresponding temperature graph is shown in FIG. 5B. As shown, there is a high and relatively narrow temperature peak near the free end of the fiber shaft.

In the frequency range of 100 kHz to 2.5 MHz, there are two physical effects that appear in the fiber shaft during its excitation for pulse durations of several seconds.

The first effect is expressed as relative over-heating of the last hot spot in the shaft near the bottom of the rod (a free boundary). In a linear model with energy losses, all hot spots along the shaft achieve the same temperature. However, during its heating, the fiber's physical properties change. The absorption rate of the fiber material increases, and the characteristic speed of sound and wavelength decrease. The change wavelength results in a shift of the hot spots towards the free boundary because the last zero power point remains on the boundary. For example, if the last peak close to the boundary is shifted by Δλ, where λ is the original wavelength (before heating) the next peak is shifted by 2Δλ, etc. As a result, the last hot spot stays nearly in its primary position during the entire pulse and achieves a relatively higher temperature. This effect does not depend on the presence or absence of the bulb.

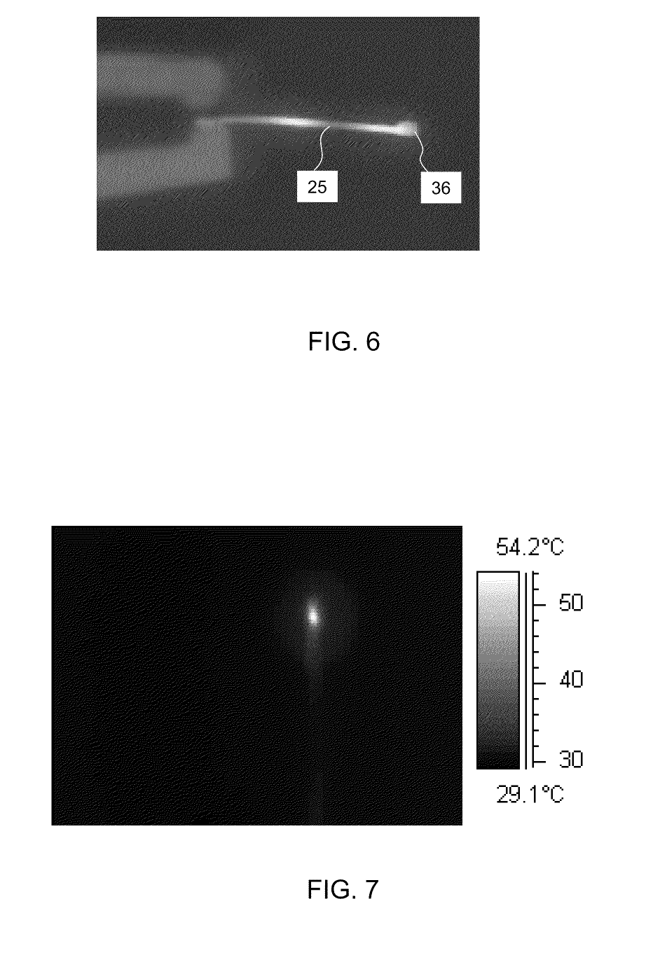

The second effect depends on the presence of the bulb at the end of the fiber shaft. This effect is more prominent when the wavelength is much longer than the length of the bulb (along the longitudinal direction of the bulb). In the solid rod model of the present example, the presence of a massive bulb at the free boundary causes a significant shift in the location of last maximal power point in the direction of the bulb. When the absorption rate of material is higher in the bulb than in the shaft, over-heating occurs in the bulb. This is demonstrated in the simulation results shown in the infrared image of FIG. 6.

The above two effects lead to over-heating of the bulb area thus facilitating generation of high temperatures in the bulb region and lower temperatures along the fiber shaft.

FIG. 7 is an infrared map showing simulations for a case in which the bulb is made of a material with higher mechanical energy absorption and lower thermal conductivity compared to the material of the shaft. As shown, such configuration produces a pure bulb effect, wherein the temperature of the fiber is low and the temperature of the bulb is high.

Following is a description of a mathematical model of longitudinal waves in a one-dimensional solid rod.

The symbols used in the following description are summarized in Table 1.

TABLE 1

| Symbol | Physical Quantity |
|---|---|
| r | radius of the rod |
| L | length of the rod |
| s | cross-sectional area of the rod |
| λ | typical wavelength |
| z | coordinate along the longitudinal axis of the rod, from 0 to L |
| p | stress in the positive z direction; |
| f | pulling force along the rod |
| s | rod crossing square |
| p | f/s |
| u | elongation in the positive z direction. |
| u_{z} | $strain in z direction {u}_{z} = \frac{\partial u}{\partial z}$ |
| E | Young modulus of the rod material |
| v | vibrovelocity |
| ρ | density of the rod material |
| c | $velocity of longitudinal waves in the rod , c = \sqrt{\frac{E}{ρ}}$ |
| η, ζ | first and second viscosity coefficients |

In the following, it is assumed that r<<λ and r<<L.

Hook's law reads:

u_{z}=p/E

Time differentiation of Hook's law:

$$\frac{\partial {u}_{z}}{\partial t} = \frac{{\partial }^{2} u}{\partial t \partial z} = \frac{1}{E} \frac{\partial p}{\partial t} ; \\ \frac{\partial }{\partial z} v = \frac{1}{E} \frac{\partial }{\partial t} p ; \\ ρ {v}^{′} = \frac{1}{{c}^{2}} \text{?} \\ \text{?} \text{indicates text missing or illegible when filed} \qquad ( EQ . 1 )$$

Newton law reads:

ρ [symbol] p′=0 (EQ. 2)

Standing Wave without Mechanical Energy Dissipation

The system of equations is:

$$\{ \begin{matrix}ρ {v}^{′} - \frac{1}{{c}^{2}} \text{?} = 0 ; \\ ρ \text{?} {p}^{′} = 0.\end{matrix} \\ \text{?} \text{indicates text missing or illegible when filed} \qquad ( EQ . 3 )$$

Source of vibration located in 0-point.

In the following, two types of sources are considered. A v-type source which outputs amplitude of vibrovelocity v₀, and a p-type source (with output amplitude of stress p₀.

(i) v-Type Source:

The boundary conditions are

v(0)=v₀ cos ωt;

p(L)=0.

The solution to EQ. 3 under these boundary conditions is:

$$\{ \begin{matrix}v = {v}_{0} \frac{sin [ k ( L - z ) μ π / 2 ]}{sin ( k L μ π / 2 )} cos ω t ; \\ p = - ρ c {v}_{0} \frac{cos [ k ( L - z ) μ π / 2 ]}{sin ( k L μ π / 2 )} sin ω t .\end{matrix} \qquad ( EQ . 4 )$$

(ii) p-Type Source:

The boundary conditions are:

p(0)=−p₀ cos ωt;

p(L)=0.

The solution to EQ. 3 under these boundary conditions is:

$$\{ \begin{matrix}v = \frac{{p}_{0}}{ρ c} \frac{cos k ( L - z )}{sin k L} sin ω t ; \\ p = - {p}_{0} \frac{sin k ( L - z )}{sin k L} cos ω t .\end{matrix} \qquad ( EQ . 5 )$$

The time averaged energy density of harmonic wave during a single period is the sum of kinetic and strain components:

$$\overline{E} = {\overline{E}}_{k} + {\overline{E}}_{s} = \frac{1}{2} ρ {\overline{v}}^{2} + \frac{1}{2} \frac{{\overline{p}}^{2}}{ρ {c}^{2}} \qquad ( EQ . 6 )$$

From EQ. 4 one obtains the average kinetic and strain energy density for the v-source:

$${\overline{E}}_{k} = \frac{1}{8} \frac{ρ {v}_{0}^{2}}{{cos}^{2} k L} [ 1 + cos 2 k ( L - z ) ] ; \\ {\overline{E}}_{s} = \frac{1}{8} \frac{ρ {v}_{0}^{2}}{{cos}^{2} k L} [ 1 - cos 2 k ( L - z ) ] . \qquad ( EQ . 7 )$$

From EQ. 5 one obtains the average kinetic and strain energy density for the p-source:

$${\overline{E}}_{k} = \frac{1}{8} \frac{{p}_{0}^{2}}{ρ {c}^{2} {sin}^{2} k L} [ 1 + cos 2 k ( L - z ) ] ; \\ {\overline{E}}_{s} = \frac{1}{8} \frac{{p}_{0}^{2}}{ρ {c}^{2} {sin}^{2} k L} [ 1 - cos 2 k ( L - z ) ] . \qquad ( EQ . 8 )$$

### Dissipation of Mechanical Energy

Internal friction (viscosity) in the rod material causes dissipation of wave energy in the form of heat.

Without loss of generality it is assumed that both the thermal conductivity and the loss factor of the rod material are small. The process may be described by a dissipative function R, where 2R determines the loss of mechanical energy per unit of time.

For an isotropic solid three-dimensional body:

$$R = {η ( {v}_{ik} - \frac{1}{3} {δ}_{ik} {v}_{ll} )}^{2} + \frac{ζ}{2} {v}_{ll}^{2} ;$$

$$i , k , l = 1 , 2 , 3 ;$$

$${v}_{ik} = \frac{\partial {v}_{i}}{\partial {x}_{k}} .$$

For a one-dimensional rod the function R can be written as:

$$R = \frac{ρ {c}^{2}}{2 ω} {ɛ ( {v}^{′} )}^{2} , \qquad ( EQ . 9 )$$

where ε is a dimensionless loss factor, ε<<1. The time averaged losses of the mechanical energy (during a period T) per unit time are:

$$2 \overline{R} = \frac{ρ {c}^{2}}{ω} ɛ \frac{1}{T} {\int }_{0}^{T} {( {v}^{′} )}^{2} d t . \qquad ( EQ . 10 )$$

For a small loss factor, the process of heating is much slower than the vibration process and at any point along the rod 2R can be written as:

$$2 R = \frac{\partial Q}{\partial τ} , \qquad ( EQ . 11 )$$

where τ is the time of heating process, and Q is the heat energy density.

From EQs. 4 and 5 one obtains for the v-source, and p-source, respectively:

$$\begin{matrix}\frac{\partial Q}{\partial τ} = \frac{1}{4} ɛω \frac{ρ {v}_{0}^{2}}{{cos}^{2} k L} [ 1 - cos 2 k ( L - z ) ] = 2 ɛω {\overline{E}}_{s} & ( EQ . 12 ) \\ \frac{\partial Q}{\partial τ} = \frac{1}{4} ɛω \frac{{p}_{0}^{2}}{ρ {c}^{2} {sin}^{2} k L} [ 1 - cos 2 k ( L - z ) ] = 2 ɛω {\overline{E}}_{s} & ( EQ . 13 )\end{matrix}$$

Therefore, the dissipated part of the standing wave energy at any point along the rod is proportional to the strain energy density of this point for any type of wave source. The factor εω is proportional to the squared frequency and to a linear combination of the two viscosity coefficients ζ and η. It is also inversely proportional to material density and the square of the sound velocity. Thus, for small dissipation and thermal conductivity, essential heating takes place only close to maximum points of the trigonometric function 1−cos 2k(L−z).

For a linear approximation of EQ. 12, the rightmost part of the equation is not dependent on the time and

$${\int }_{0}^{Δ T} \frac{\partial Q}{\partial τ} d τ = 2 ɛω {\overline{E}}_{s} Δ T \qquad ( EQ . 14 )$$

where ΔT is the duration of heating.

In a one-dimensional rod, the energy flux J from the source along the rod is described by the equation:

$$\frac{\partial J}{\partial z} = - \frac{\partial Q}{\partial τ} \qquad ( EQ . 15 )$$

From EQs. 12 and 13 one obtains have J(z) for the v-source and p-source, respectively:

$$\begin{matrix}J ( z ) = \frac{ɛωρ {v}_{0}^{2}}{4 {cos}^{2} k L} [ ( L - z ) - \frac{1}{2 k} sin 2 k ( L - z ) ] & ( EQ . 16 ) \\ J ( z ) = \frac{ɛω {p}_{0}^{2}}{4 ρ {c}^{2} {sin}^{2} k L} [ ( L - z ) - \frac{1}{2 k} sin 2 k ( L - z ) ] . & ( EQ . 17 )\end{matrix}$$

The integration constant was found by the present inventor using the boundary condition J(L)=0.

EQs. 16 and 17 describe J(z) as a monotone decreasing positive function with a maximal value at z=0.

The source power Wₛ is calculated by multiplying J(0) by the cross-sectional area of the rod.

Wₛ=J(0)s. (EQ. 18)

Substituting J(0) one obtains:

$${W}_{s} = J ( 0 ) s = s {\int }_{L}^{0} \frac{\partial Q}{\partial τ} d z . \qquad ( EQ . 19 )$$

The loss factor ε and the speed of sound c are weakly dependent of τ. This relationship can be described by respectively changing the constants (ε, c, k) in (EQs. 12 and 13 by the functions ε=ε(τ); c=c(τ); k=ω/c(τ). All expressions remain in their linear form.

The change in wavelength shifts the hot spots so that they approach to the free boundary if the speed of sound in the rod material increases with temperature, and retreat from the free boundary if the speed of sound decreases with temperature. For example, if the last peak (close to the boundary) is shifted by Δλ, the next peak is shifted by 2Δλ, and so no. As a result, the last hot spot stays nearly in its primary position during the entire pulse and achieves a relatively higher temperature. This is a phenomenon of energy flux from vibration source to opposite boundary in a quasi-standing wave. This effect typically appears when there is a change in the speed of sound in the rod material during the pulse and the thermal conduction of the rod material is relatively low. It was found by the present inventor the effect does is not dependent of weakly dependent on the type of boundary condition. The constant parameter of the source is the stress amplitude p₀ (for p-type source) or the vibrovelocity amplitude v₀ (for a v-type source), but not the z-phase or source power. Thus, during the peaks movement their amplitude can change. This phenomenon is known as the parametric resonance phenomenon, wherein there is a parameter that is varying (e.g., periodically, but may also be any variation) such that the system is excited. In the context of the present embodiments, the varying parameter is the speed of sound.

The effect of bulb heating can be described mathematically as the replacement of the boundary condition at z=L from p=0 to a mass type impedance boundary condition. The boundary impedance is proportional to the frequency and mass of the bulb per unit length. This change causes the last heating peak to shift closer to the bulb.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for generating heat at a target location, comprising:
a fiber device having a distal end and a proximal end;
an acoustic wave generator for generating acoustic waves; and
an acoustic coupler for coupling acoustic energy carried by said acoustic waves into said distal end such that heat is generated at said distal end.

2. The system according to claim 1, further comprising an acoustic amplifier configured for amplifying amplitude of said acoustic waves prior to said coupling of said acoustic energy into said fiber device.

3. The system according to claim 2, wherein said acoustic amplifier comprises an acoustic horn.

4. The system according to claim 1, wherein said acoustic coupler comprises a container filled with an impedance matching medium.

5. The system according to claim 4, wherein said container comprises a first end and a second end, wherein said acoustic wave generator is coupled to said container at said first end, and wherein said second end comprises an opening which receives said proximal end of said fiber device.

6. The system according to claim 5, wherein a height of said at least one opening is approximately an integer multiplication of half a wavelength of said ultrasound waves.

7. The system according to claim 1, wherein said acoustic coupler comprises a mechanical gripper having gripping ends biasable towards each other, wherein said acoustic wave generator induces vibrations at least at one of said gripping ends, and wherein said fiber device is positioned between said gripping ends to receive said vibrations.

8. The system according to claim 7, wherein said at least one gripping end is sufficiently flexible and sufficiently elastic such that amplitude of said vibrations is larger at said at least one gripping end than at said acoustic wave generator.

9. The system according to claim 7, wherein said acoustic coupler comprises an acoustic horn and wherein said at least one gripping end and said acoustic horn are made of the same material.

10. The system according to claim 9, wherein said at least one gripping end is an integral extension of said acoustic horn.

11. A method of generating heat at a target location, comprising: guiding a distal end of a fiber device to said target location and coupling acoustic energy into said fiber device at a proximal end of said fiber device such as to generate heat at said distal end, thereby generating heat at said target location.

12. The method according to claim 11, wherein said heat is generated at an amount being sufficient to treat a target tissue at said target location.

13. The method according to claim 12, wherein said target tissue is selected from the group consisting of a tumor, a lesion and an inflammatory tissue.

14. (canceled)

15. (canceled)

16. The method according to claim 12, wherein said target tissue is a skin tissue.

17. The method according to claim 12, wherein said target tissue is part of an internal organ.

18. The method according to claim 12, wherein said target tissue is a blood vessel clot.

19. The method according to claim 12, wherein said guiding is via a minimally invasive procedure.

20. The method according to claim 12, wherein said guiding is during an invasive procedure.

21. The method according to claim 11, further comprising amplifying amplitude of acoustic waves carrying said acoustic energy prior to said coupling of said acoustic energy into said fiber device.

22. The system according to claim 1, wherein said fiber device comprises a bulb at said distal end.
